(19) United States
(12) Patent Application Publication
(10) Pub. No.: US 2015/0126611 A1
(43) Pub. Date: May 7, 2015

(54) 3,3-DIPHENYL-N-(1-PHENYLETHYL) PROPAN-1-AMINE: AS A NEW SELECTIVE LIGAND OF THE SIGMA-1 RECEPTORS, WITH ANTI-APOPTOTIC (CYTOPROTECTIVE) PROPERTIES AND PROTOTYPICAL ANTICANCER ACTIVITY

(71) Applicant: VAMVAKIDES Alexandre, Ymittos Attikis (GR)

(72) Inventor: Alexandre Vamvakides, Ymittos Attikis (GR)

(21) Appl. No.: 14/406,271

(22) PCT Filed: Jan. 8, 2013
(86) PCT No.: PCT/GR2013/000001

§ 371 Date: Dec. 8, 2014

(30) Foreign Application Priority Data
Jan. 10, 2012 (GR) 20120100013

(51) Int. Cl. C07C 211/27

(52) U.S. Cl. CPC C07C 211/27
USPC 514/648; 564/316

(57) ABSTRACT

3,3-diphenyl-N-(1-phenylethyl) propan-1-amine (fendiline): as a new selective ligand of the sigma-1 receptors, with anti-apoptotic (cytoprotective) properties and prototypical anticancer activity. This invention concerns the prototypical profile of selective sigma-1 ligand and the putative therapeutical properties of the compound 3,3-diphenyl-N-(1-phenylethyl) propan-1-amine (DPPA) and its pharmaceutically acceptable salts, with cytoprotective activity, more specifically for neurons against the neurodegenerative diseases (e.g. Alzheimer's disease, Huntington's disease, Parkinson's disease) via their anti-apoptotic properties induced by their selective sigma-1 agonism. Concerning the cancer cells, DPPA exhibited pro-apoptotic properties associated with neuroprotective activity originating a prototypical anticancer profile consisting in synergistical association with the clinically used anticancer drugs with its analgesic and neuroprotective activities antagonizing the neuropathic pain induced by the later.

### CROSS-REFERENCE TO RELATED APPLICATION

This application is a U.S. national phase application under 35 U.S.C. §371 based upon International Application No. PCT/GR2013/000001 filed on Jan. 8, 2013. Additionally, this U.S. national phase application claims the benefit of priority of International Application No. PCT/GR2013/000001 filed on Jan. 8, 2013, and Greece Application No. 20120100013 filed on Jan. 10, 2012. The entire disclosures of the prior applications are incorporated herein by reference. The international application was published on Jul. 18, 2013 under Publication No. WO 2013/104933 A1.

### BACKGROUND OF THE INVENTION

### FIELD OF THE INVENTION

The present invention constitutes an up to date version of the Greek Patent no 1007853/15-03-2013 (Application, no 20120100013/10-01-2012, Priority date Jan. 10, 2012) (WO 2013/104933). It concerns the prototypical profile of sigma-1 selective ligand and the new putative therapeutical properties of 3,3-diphenyl-N-(1-phenylethyl) propan-1-amine (DPPA) characterized by 40 to 60 fold higher affinities for sigma-1 vs. sigma-2 receptors with median concentrations of binding (IC50) being 14 to 18 nanoMoles for sigma-1 receptors and 730 to 850 nanoMoles for sigma-2 receptors.

### SUMMARY OF THE INVENTION

It concerns the prototypical profile of selective sigma-1 ligand and the putative therapeutical properties of the compound 3,3-diphenyl-N-(1-phenylethyl) propan-1-amine (DPPA) and its pharmaceutically acceptable salts, with cytoprotective activity, more specifically for neurons against the neurodegenerative diseases, via their anti-apoptotic properties induced by their selective sigma-1 agonism. In particular, concerning the Alzheimer's disease (AD), the prototypical neuroprotection (M2[−] and σ1[+]) of DPPA allowed the putative valorization of IAChEases as therapeutic agents against AD. Concerning the cancer cells, DPPA exhibited pro-apoptotic properties associated with neuroprotective activity originating a prototypical anticancer profile consisting in synergistical association with the clinically used anticancer drugs with its analgesic and neuroprotective activities antagonizing the neuropathic pain induced by the later.

### DETAILED DESCRIPTION OF THE INVENTION

The present invention concerns the prototypical profile of sigma-1 selective ligand and the new putative therapeutical properties of 3,3-diphenyl-N-(1-phenylethyl) propan-1-amine (DPPA) characterized by 40 to 60 fold higher affinities for sigma-1 vs. sigma-2 receptors with median concentrations of binding (IC50) being 14 to 18 nanoMoles for sigma-1 receptors and 730 to 850 nanoMoles for sigma-2 receptors. The high affinity of DPPA for the site-2 of the sodium channels with IC50=290 nanoMoles participates in the prototypical anticancer and anti-metastatic activities of DPPA associated with analgesic activity against the neuropathic pain, induced by the clinically used anticancer drugs (Taxanes, Platins, Vincristin), at 1.0 to 50.0 mg/kg, per os (po), in mice. More important- at lower doses: 0,01 to 1,00 mg/kg, intraperitoneal (ip) in mice—the cytoprotective activity of DPPA obtained by antagonism of the endoplasmic reticulum stress (ER stress) and amelioration of the cytoprotective autophagic processus of the cells and, more specifically, of neurons (in the neurodegenerative diseases and eldery). By its highly selective sigma-1 agonism (σ1[+]), DPPA activated the anti-apoptotic pathways (stimulation of the anti-apoptotic proteins Bcl-2, Bcl-xL) and restored the mitochondrial function of the neurons preventing the oxidative stress and apoptosis (neuronal death). The above was clearly demonstrated by the capacity of DPPA to antagonize the mitochondrial toxicity of pentylenetetrazole (PTZ) by increasing the latency of lethality of PTZ (130 mg/kg, ip), in mice, by 5, 10 and 20 fold at, respectively, 3, 10 and 30 mg/kg (po) of DPPA. These cytoprotective activities of DPPA were completed, specifically in neurons, by inhibiting their entry in the cell cycle, as this entry is always fatal for neurons. In contrast, concerning the cancer cells, the above inhibition triggered the apoptotic processus and their death. The highly selective sigma-1 activity of DPPA and its modulation of the sodium channels are implicated in the above effects originating the protection of neurons and the death of cancer cells.

The experimental results on mice (antagonism by DPPA of the scopolamine, dizocilpine and amyloids Aβ25-35 induced amnesias) demonstrated the putative antiamnesic and anti-neurodegenerative activity of DPPA and in vitro experiments with cancer cells and normal cells have demonstrated the anticancer activity of DPPA on colon, prostate, ovarian, pancreas and hepatic cancer cells.

The aforementioned experimental data and the pharmacological properties of DPPA clearly addressed the therapeutic potential against the pathophysiological causes of the reported pathologies. Therefore, in the following claims, the proposed pharmaceutical uses are therapeutical and not simply addressed to the symptoms without stopping or delaying the evolution of the diseases.

Indeed, concerning the cytodegenerative diseases and more specifically the neurodegenerative diseases (Alzheimer, Huntington, Parkinson or Multiple Sclerosis), DPPA, in contrast to the symptomatic treatments used currently against the above diseases, antagonizes their pathophysiological causes ie., the endocellular biochemical stress and, concerning the neurons, also their fatal entry in the cell cycle. Of particular interest was the action of DPPA when it was coadministered with the inhibitors of the acetylcholinesterases (IAChEases) which are used as symptomatic drugs (principally Rivastigmine, Donepezil and Galantamine) against the Alzheimer's Disease (AD). The antiamnesic action of DPPA—in the experimental protocols (Y-maze and step-through) of neurodegenerative amnesias, obtained by intracerebral injection of soluble amyloids Aβ25-35 in mice (which reproduced the endoneuronal stress of AD)—is outstanding (higher than the sum of the antiamnesic effects of DPPA and IAChEases (typical examples Rivastigmine or Donepezil) when administered alone). Indeed, DPPA, via its antagonistic action on the M2 muscatinic autoreceptors of the brain cholinergic neurons (M2[−]), abolished the pro-neurodegenerative agonistic activity of the IAChEases on the hyperactivated (by AD or by the soluble Aβ25-35) cerebral M2 muscarinic autoreceptors, originating a new pharmacological approach (M2[−]) and (σ1[+]) against the neurodegenerative effects of Aβ25-35 in AD and also against the deleterious effect of the agonistic action of IAChEases on the M2 muscarinic brain autoreceptors, validating these drugs (IAChEases) as putative therapeutic agents against AD.

The above neuroprotective activity of DPPA is completed by the prevention of the severe cholinergic adverse effects (nausea, vomiting, diarrhea, bradycardia) of IAChEases via the antagonistic effects of DPPA (from 0.1 to 10 mg/kg, ip, in mice) on the M2 and M3 muscarinic receptors, in the periphery, therefore permitting the use of higher and more efficient doses of IAChEases against AD.

Evidence, that the spectacular antiamnesic action of the coadministration of DPPA+IAChEases is the consequence of the aforementioned neuroprotective effects (M2[−] and (σ1[+]), was obtained by the absence of any, even simple, synergistical antiamnesic effect of the coadministration of DPPA+IAChEases in the scopolamine induced amnesia, on mice (a neuropharmacological model of amnesia, in which are not implicated neurodegenerative phenomena). Consequently, the high neuroprotective antiamnesic effect of DPPA+IAChEases imposes, for its pharmacological characterization, the wording of “theurapeutic potentiation” which, concerning the AD, has the signification of therapeutic protection against the fatal evolution of AD with the consequent amelioration of the quality of life of patients, the suppression of the severe adverse effects of the IAChEases and, more importantly, the theurapeutic protection by stabilization of AD in the most early steps of the disease. Indeed, only the symptoms (partial) treatment of AD, without any protection against its fatal evolution, has been, historically, registered by monotherapy with IAChEases, during the past 3 decades.

In vivo experiments with the appropriate protocols, on mice: forced swimming (Porsolt), Open field and Elevated plus maze, with DPPA, demonstrated the prototypical antidepressive action which is associated with neuroprotective effects (confirmed by the neuroprotective antagonism of the neurotoxic effect of Pentylenetetrazole (PTZ) on the mitochondria of neurons and also by the afore mentioned anti-Aβ25-35 activity). The anti-immobility properties of DPPA in the Porsolt (significative at more than 10 mg/kg, po, in mice) have been mentioned in previous studies. However, the combination of these properties with anti-apoptotic and neuroprotective effects is a new inventive approach of therapeutic and not only symptomatic confrontation of the disease, as there is mounting evidence that depression is a neurodegenerative disease, via endoneuronal biochemical stress and apoptosis of neurons.

The anticancer activity of DPPA was studied in vitro and in vivo by inoculation of cancer cells in immunosuppressed SCID mice. While its anticancer activity, in agreement with previous studies, is lower than those of the clinically used anticancer drugs, the coadministration of DPPA with the later (and more specifically with Taxanes, Platins and Vinca alkaloids) is a new and important inventive approach as, in the coadministration of DPPA with the above anticancer drugs, except the obtained synergistical anticancer and antimetastatic effects, DPPA antagonized the pathophysiological causes (endoneuronal biochemical stress and neuronal degenerative injuries) of the neurotoxicity of anticancer drugs. Moreover, even when the neuropathic pain, induced by the anticancer drugs was installed, DPPA antagonized it, by inhibition of the hypersensitive neurons injured by the neurotoxicity of the above anticancer drugs.

Finally, in the experimental protocol of neuropathic pain by sensibilisation with Paclitaxel and evaluation of the neuropathic pain by injection of formalin in the hind paw of mice, the analgesic effect of DPPA was significative from 1.0 mg/kg (po) and was spectacular at higher doses (25 to 75 mg/kg, po). Indeed, the Paclitaxel+DPPA treated mice were less sensible to formalin than the control mice not treated with Paclitaxel.

1-8. (canceled)

9. A compound comprising an effective amount of a compound selected from the group consisting of 3,3-diphenyl-N-(1-phenylethyl) propan-1-amine, a pharmaceutically acceptable salt thereof, and combinations thereof, said compound being as prototypical selective ligands of sigma-1 receptors with anti-apoptotic activity on normal cells and pro-apoptotic properties on cancer cells.

10. A pharmaceutical composition comprising an effective amount of a compound, and at least one pharmaceutically acceptable excipient, said compound being selected from the group consisting of 3,3-diphenyl-N-(1-phenylethyl) propan-1-amine, a pharmaceutically acceptable salt thereof, and combinations thereof, said compound being as prototypical selective ligands of sigma-1 receptors with anti-apoptotic activity on normal cells and pro-apoptotic properties on cancer cells.

11. A method of using a compound comprising an effective amount of a compound selected from the group consisting of 3,3-diphenyl-N-(1-phenylethyl) propan-1-amine, a pharmaceutically acceptable salt thereof, and combinations thereof, said compound being as prototypical selective ligands of sigma-1 receptors with anti-apoptotic activity on normal cells and pro-apoptotic properties on cancer cells, said method of using said compound for preparation of pharmaceuticals.

12. The method of using the compound according to claim 11, wherein said pharmaceuticals with cytoprotective activity against cytodegenerative and neurodegenerative processes in one condition selected from the group consisting of Alzheimer's, Huntington's, Parkinson's, and Multiple Sclerosis, at doses 3 to 30 mgs/day orally.

13. The method of using the compound according to claim 11, wherein said pharmaceuticals with antidepressive activity associated with neuroprotection against the pathological apoptosis of neurons in depression, at doses of 30 to 150 mgs/daily, per os.

14. The method of using the compound according to claim 11, wherein said pharmaceuticals being used against cholinergic adverse effects of IAChEases, in a symptomatic treatment of AD, by an antagonism of M2 and M3 muscarinic receptors exerced by said compound.

15. The method of using the compound according to claim 11, wherein said pharmaceuticals being used for valorization of IChEases as therapeutic agents against evolution of AD, by an antagonism of a pre-synaptic muscarinic M2 autoreceptors completed with a sigma-1 selective agonism exerced by said compound.

16. The method of using the compound according to claim 11, wherein said pharmaceuticals being provided at doses 3 to 30 mgs/day orally against adverse cholinergic effects of Donepezil provided orally at doses selected from the group consisting of 5, 10 and 23 mgs/day, in a symptomatic treatment of AD, by antagonism of M2 and M3 muscarinic receptors.

17. The method of using the compound according to claim 11, wherein said pharmaceuticals being provided at doses 3 to 30 mgs/day orally for valorization of Donepezil provided orally at doses selected from the group consisting of 5, 10 and 23 mgs/day as therapeutic drug against evolution of AD, by antagonism of presynaptic muscarinic M2 autoreceptors completed with sigma-1 selective agonism.

18. The method of using the compound according to claim 11, wherein said pharmaceuticals being provided at doses 3 to 30 mgs/day orally against adverse cholinergic effects of Rivastigmine provided at doses selected from the group consisting of orally at 6 to 12 mgs/day, and transdermal patch at 4.6, 9.5 or 13.3 mgs/day, in symptomatic treatment of AD, by antagonism of M2 and M3 muscarinic receptors.

19. The method of using the compound according to claim 11, wherein said pharmaceuticals being provided at doses 3 to 30 mgs/day orally for valorization of Rivastigmine provided at doses selected from the group consisting of orally at 6 to 12 mgs/day, and transdermal patch at 4.6, 9.5 or 13.3 mgs/day, as therapeutic drug against evolution of AD, by antagonism of presynaptic muscarinic M2 autoreceptors completed with selective sigma-1 agonism.

19. The method of using the compound according to claim 11, wherein said pharmaceuticals being provided at doses 3 to 30 mgs/day orally against adverse cholinergic effects of Galantamine provided at doses selected from the group consisting of orally at 4, 8 or 12 mgs/day, tablets at 4, 8 or 12 mgs/day, and capsules at 8, 16 or 24 mgs/day, in symptomatic treatment of AD, by antagonism of M2 and M3 muscarinic receptors.

20. The method of using the compound according to claim 11, wherein said pharmaceuticals being provided at doses 3 to 30 mgs/day orally for valorization of Galantamine provided at doses selected from the group consisting of orally at 4, 8 or 12 mgs/day, tablets at 4, 8 or 12 mgs/day, and capsules at 8, 16 or 24 mgs/day, as therapeutic drug against evolution of AD, by antagonism of presynaptic muscarinic M2 autoreceptors completed with selective sigma-1 agonism.

21. The method of using the compound according to claim 11, wherein said pharmaceuticals having analgesic properties exerced against neuropathic pain, at doses of 30 to 300 mgs/day, orally.

22. The method of using the compound according to claim 11, wherein said pharmaceuticals which act synergistically with clinically used anticancer drugs and protect neurons against toxicity of the later, at doses of 30 to 150 mgs/day, orally.

23. The method of using the compound according to claim 11, wherein said pharmaceuticals which act synergistically with clinically used anticancer drugs and antagonize neuropathic pain induced by the later, at doses of 30 to 300 mgs/day, orally, said anticancer drugs selected from the group consisting of Taxanes, Platins and Vincristine.
